(19) United States
(12) Patent Application Publication
(10) Pub. No.: US 2011/0165198 A1
(43) Pub. Date: Jul. 7, 2011

(54) METHOD FOR REDUCING LIPID ABSORPTION BY AN ANIMAL

(76) Inventors: Delphine Tissot-Favre, Webster Grove, MO (US); Frederic Destaillats, Servion (CH); Yuanlong Pan, Chesterfield, MO (US); Fabiola Dionisi, Epalinges (CH); Cristina Cruz-Hernandez, Epalinges (CH); Clementine Thabuis, Lille (FR); Jean-Charles Martin, Marseille (FR)

(21) Appl. No.: 12/736,787

(22) PCT Filed: May 15, 2009
(86) PCT No.: PCT/US09/03068

§ 371 Date: Mar. 7, 2011

Related U.S. Application Data
(60) Provisional application No. 61/128,116, filed on May 19, 2008.

(51) Int. Cl. A61K 9/00; A61K 31/16; C07C 233/20; C07C 233/09; A61P 3/06; A61P 1/14; A61P 3/00; A61K 35/66; G09B 23/28

(52) U.S. Cl. 424/400; 514/627; 554/66; 554/35; 424/93.1; 434/262

(57) ABSTRACT

The invention provides methods for using fatty acid alkanolamides for reducing lipid absorption from the intestine of an animal. Reducing lipid absorption results in fewer lipids available for an animal to convert to body fat. Converting fewer lipids to body fat means that the animal reduces body fat, body weight, and/or body weight gain when consuming an equivalent amount of lipids without the fatty acid alkanolamides.

### CROSS REFERENCE TO RELATED APPLICATIONS

This application is a national stage application under 35 U.S.C. §371 of PCT/US2009/003068 filed May 15, 2009, which claims priority to U.S. Provisional Application Ser. No. 61/128,116 filed May 19, 2008, the disclosures of which are incorporated herein by reference.

### BACKGROUND OF THE INVENTION

1. Field of the Invention

The invention relates generally to methods for reducing lipid absorption and particularly to methods for using fatty acid alkanolamides for reducing lipid absorption by an animal.

2. Description of Related Art

Fatty acid alkanolamides are compounds containing a fatty acid moiety linked to alkanolamine. Fatty acid ethanolamides are compounds containing a fatty acid moiety linked to ethanolamine. These compounds are part of a family of lipids that are generally found in plant and animal tissues. Numerous Fatty acid alkanolamides and Fatty acid ethanolamides are known in the art, e.g., the compounds disclosed in U.S. Pat. No. 6,911,474 and WO05115370A2. Two common Fatty acid ethanolamides are anandamide (N-arachidonoyl-ethanolamine) and N-oleoyl-ethanolamide.

U.S. Pat. No. 6,911,474, US20050154064A1, and US2005187254A1 disclose using fatty acid alkanolamides and their homologs and analogs to reduce body fat, reduce food consumption, and modulate lipid metabolism. WO08040756A1 discloses the use of anandamide for the manufacture of a nutraceutical for oral intake preferably a medicament for reducing appetite, giving a satiety effect, preventing or reducing inflammatory bowel disease, or preventing or reducing irritable bowel syndrome. However, the references do not disclose that such compounds affect lipid absorption in an animal.

In general, fatty acid alkanolamides are known to affect appetite and modulate lipid metabolism. As a result, an animal with a reduced appetite consumes less dietary lipids and therefore reduces body fat. Similarly, an animal with an enhanced lipid metabolism tends to utilize more lipids for basic metabolic functions and therefore reduces body fat. This loss of body fat generally results in a reduction in body weight or a reduction in weight gain for an animal. While these mechanisms may be useful for reducing body fat in an animal, there is a need for new methods for reducing body fat, reducing body weight, and reducing body weight gain in an animal.

### SUMMARY OF THE INVENTION

It is, therefore, an object of the present invention to provide methods for reducing lipid absorption by an animal.

It is another object of the invention to provide methods for reducing body fat in an animal.

It is a further object of the invention to provide methods for reducing body weight of an animal.

It is another object of the invention to provide methods for reducing body weight gain by an animal.

It is another object of the invention to provide kits suitable for administering fatty acid alkanolamides to an animal.

One or more of these or other objects are achieved using novel methods for reducing lipid absorption by an animal. The methods comprise administering one or more fatty acid alkanolamides to an animal in an amount effective for reducing lipid absorption by the animal. Reducing lipid absorption results in fewer lipids available for an animal to convert to body fat. Converting fewer lipids to less body fat means that the animal reduces body fat, body weight, or body, weight gain when consuming an equivalent amount of lipid containing food.

Other and further objects, features, and advantages of the present invention will be readily apparent to those skilled in the art.

### DETAILED DESCRIPTION OF THE INVENTION

### Definitions

The term “N-oleoyl-ethanolamide” means N-oleoyl-ethanolamide and its analogs, homologs, precursors, prodrugs, or combination thereof that have an affect on lipid absorption.

The term “fatty acid ethanolamides” means one or more fatty acid ethanolamides and their analogs, homologs, precursors, prodrugs, or combination thereof that have an affect on lipid absorption, including N-oleoyl-ethanolamide.

The term “fatty acid alkanolamides” means one or more fatty acid alkanolamides and their analogs, homologs, precursors, prodrugs, or combination thereof that have an affect on lipid absorption, including fatty acid ethanolamides and N-oleoyl-ethanolamide.

The term “animal” means any animal that could benefit from a reduction in lipid absorption, including human, avian, bovine, canine, equine, feline, hicrine, lupine, murine, ovine, or porcine animals.

The term “companion animal” means domesticated animals such as cats, dogs, rabbits, guinea pigs, ferrets, hamsters, mice, gerbils, horses, cows, goats, sheep, donkeys, pigs, and the like.

The term “effective amount” means an amount of a compound, composition, medicament, or other material that is effective to achieve a particular physiological or biological result. For the present invention, such results include, but are not limited to, one or more of reducing lipid absorption, reducing body fat, reducing body weight, and reducing body weight gain in an animal.

The term “food” or “food product” or “food composition” means a product or composition that is intended for ingestion by an animal and that provides nutrition to the animal.

The term “mg/kg/bw” means milligrams per kilogram of body weight and the term “g/kg/bw” means grams per kilogram of body weight.

The term “dietary supplement” means a product that is intended to be ingested in addition to a normal animal diet. Dietary supplements may be in any form, e.g., solid, liquid, gel, tablet, capsule, powder, and the like. Preferably they are provided in convenient dosage forms, e.g., in sachets. Dietary supplements can be provided in bulk consumer packages such as bulk powders, liquids, gels, or oils. Similarly such supplements can be provided in bulk quantities to be included in other food items such as snacks, treats, supplement bars, beverages, and the like.

The term “weight control agent” means any compound, composition, or drug useful for reducing lipid absorption or for reducing body fat, reducing body weight, or reducing body weight gain in an animal, other than the fatty acid alkanolamides of the present invention. Such agents include, but are not limited to, chitosan and related compounds such as those disclosed in U.S. Pat. No. 4,223,023 that reduce the absorption of lipids; oxetanoe derivatives such as those disclosed in U.S. Pat. No. 6,348,492; and synergistic combinations of psyllium and chitosan such as those disclosed in U.S. Pat. No. 6,506,420.

The term “in conjunction” means that fatty acid alkanolamides, weight control agents, or other compounds or compositions of the present invention are administered to an animal (1) together in a food composition, dietary supplement, or other composition or (2) separately at the same or different frequency using the same or different administration routes at about the same time or periodically. “Periodically” means that a weight control agent or other composition is administered on a dosage schedule acceptable for a specific agent or composition. “About the same time” generally means that the fatty acid alkanolamides, weight control agents, or other compositions are administered at the same time or within about 24 hours of each other. “In conjunction” specifically includes administration schemes wherein a weight control agent or other composition is administered for a prescribed period and the fatty acid alkanolamides are administered indefinitely, e.g., in a food composition.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of individual fatty acid alkanolamides and food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The dosages expressed herein are in milligrams per kilogram of body weight per day (mg/kg/day) unless expressed otherwise.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the invention provides methods for reducing lipid absorption by an animal. The methods comprise administering fatty acid alkanolamides to the animal in an amount effective for reducing lipid absorption by the animal.

In another aspect, the invention provides methods for reducing body fat in an animal. The methods comprise administering fatty acid alkanolamides to the animal in an amount effective for reducing lipid absorption by the animal.

In another aspect, the invention provides methods for reducing body weight of an animal. The methods comprise administering fatty acid alkanolamides to the animal in an amount effective for lipid absorption by the animal.

In another aspect, the invention provides methods for reducing body weight gain of an animal. The methods comprise administering fatty acid alkanolamides to the animal in an amount effective for reducing lipid absorption by the animal.

The inventions are based upon the discovery that fatty acid alkanolamides are effective for reducing lipid absorption from the intestine by an animal. Fatty acid alkanolamides are known to affect body fat, body weight, and body fat gain by reducing lipid consumption and by increasing lipid metabolism, i.e., reducing food intake by a mechanism where absorbed lipids affect satiety and increasing the metabolic use of absorbed lipids, respectively. In contrast, the present invention reduces the absorption of lipids from the intestine of an animal. The lipids do not get absorbed and therefore cannot affect satiety or be used in metabolic processes. Reducing lipid absorption means that the some lipids that would otherwise be absorbed by the animal do not pass from the intestine into the animal. These lipids pass through the animal and are excreted in the feces. Reducing lipid absorption results in fewer lipids available for an animal to convert to body fat. Converting fewer lipids to less body fat means that the animal reduces body fat, body weight, or body weight gain when consuming an equivalent amount of lipids, particularly lipid containing foods. When lipids or materials containing lipids are consumed by an animal in conjunction with one or more fatty acid alkanolamides, less lipid is absorbed by the animal than would have been absorbed by the animal if the lipids or materials containing lipids had been consumed without the fatty acid alkanolamides.

In one embodiment, the fatty acid alkanolamides are fatty acid ethanolamides. In another, the fatty acid alkanolamides are N-acyl fatty acid ethanolamides. In a further, the fatty acid alkanolamide is N-oleoyl-ethanolamide. In another, the fatty acid alkanolamide is (Z)—(R)-9-octadecenamide, N-(2-hydroxyethyl, 1-methyl).

In various embodiments, the fatty acid alkanolamides are hydrolysis-resistant fatty acid alkanolamide analogs, hydrolysis-resistant fatty acid ethanolamide analogs, or hydrolysis-resistant N-oleoyl-ethanolamide analogs such as (Z)—(R)-9-octadecenamide, N-(2-hydroxyethyl,1-methyl).

Fatty acid alkanolamides are administered to an animal in any amount effective for reducing lipid absorption from the intestine by the animal. Generally, fatty acid alkanolamides are administered to an animal in amounts of from about 0.1 to about 1500 mg/kg/day, preferably from about 1 to about 1000 mg/kg/day, most preferably from about 5 to about 500 mg/kg/day. Amounts of about 100 mg/kg/day are preferred.

Fatty acid alkanolamides can be administered to the animal in any suitable form using any suitable administration route. For example, fatty acid alkanolamides can be administered in a fatty acid alkanolamides composition, in a food composition, in a dietary supplement, in a pharmaceutical composition, in a nutraceutical composition, or as a medicament. Similarly, fatty acid alkanolamides can be administered using any suitable administration route, e.g., orally. Preferably, fatty acid alkanolamides are administered orally to an animal as a dietary supplement or as an ingredient in a food composition.

In a preferred embodiment, fatty acid alkanolamides are administered to an animal as an ingredient in a food composition suitable for consumption by an animal, including humans and companion animals such as dogs and cats. Such compositions include complete foods intended to supply the necessary dietary requirements for an animal or food supplements such as animal treats.

In various embodiments, food compositions such as pet food compositions or pet treat compositions comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof.

The food compositions may further comprise from about 5% to about 40% fat, generally with about 5 to 50% of energy represented by lipids. Examples of suitable fats include animal fats and vegetable fats. Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used.

The food compositions may further comprise from about 15% to about 60% carbohydrate. Examples of suitable carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

The moisture content for such food compositions varies depending on the nature of the food composition. The food compositions may be dry compositions (e.g., kibble), semi-moist compositions, wet compositions, or any mixture thereof. In a preferred embodiment, the composition is a complete and nutritionally balanced pet food. In this embodiment, the pet food may be a “wet food”, “dry food”, or food of “intermediate moisture” content. “Wet food” describes pet foods that are typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one preferred embodiment, the compositions have moisture content from about 5% to about 20%. Dry food products include a variety of foods of various moisture contents, such that they are relatively shelf-stable and resistant to microbial or fungal deterioration or contamination. Also preferred are dry food compositions that are extruded food products such as pet foods or snack foods for either humans or companion animals.

The food compositions may also comprise one or more fiber sources. The term “fiber” includes all sources of “bulk” in the food whether digestible or indigestible, soluble or insoluble, fermentable or nonfermentable. Preferred fibers are from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the present invention to the immune system of an animal.

In some embodiments, the ash content of the food composition ranges from less than 1% to about 15%, preferably from about 5% to about 10%.

In a preferred embodiment, the composition is a food composition comprising fatty acid alkanolamides and from about 15% to about 50% protein, from about 5% to about 40% fat, from about 5% to about 10% ash content, and having a moisture content of about 5% to about 20%. In other embodiments, the food composition further comprises prebiotics or probiotics as described herein. In other embodiments, the food composition further comprises weight control agents as described herein.

When administered in a food composition, fatty acid alkanolamides comprises from about 1 to about 40% of the food composition, preferably from about 3 to about 30%, more preferably from about 5 to about 20%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, or 40%.

In another embodiment, fatty acid alkanolamides are administered to an animal in a dietary supplement. The dietary supplement can have any suitable form such as a gravy, drinking water, beverage, yogurt, powder, granule, paste, suspension, chew, morsel, treat, snack, pellet, pill, capsule, tablet, sachet, or any other suitable delivery form. The dietary supplement can comprise fatty acid alkanolamides and optional compounds such as vitamins, preservatives, probiotics, prebiotics, and antioxidants. This permits the supplement to be administered to the animal in small amounts, or in the alternative, can be diluted before administration to an animal. The dietary supplement may require admixing with a food composition or with water or other diluent prior to administration to the animal. When administered in a dietary supplement, fatty acid alkanolamides comprises from about 1 to about 80% of the supplement, preferably from about 3 to about 50%, more preferably from about 5 to about 40%.

In another embodiment, fatty acid alkanolamides are administered to an animal in a pharmaceutical or nutraceutical composition. The pharmaceutical composition comprises fatty acid alkanolamides and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals. When administered in a pharmaceutical or nutraceutical composition, fatty acid alkanolamides comprises from about 1 to about 90% of the composition, preferably from about 3 to about 60%, more preferably from about 5 to about 50%.

In one embodiment, the fatty acid alkanolamides are administered in conjunction with one or more weight control agents. In another, the fatty acid alkanolamides are administered in conjunction with one or more prebiotics or probiotics. In a further, the fatty acid alkanolamides are administered in conjunction with one or more weight control agents and one or more prebiotics or probiotics.

In another aspect, the invention provides compositions comprising fatty acid alkanolamides in an amount effective for reducing lipid absorption by an animal. The compositions contain fatty acid alkanolamides in amounts sufficient to administer fatty acid alkanolamides to an animal in amounts of from about 0.1 to about 1500 mg/kg/day, preferably from about 1 to about 1000 mg/kg/day, most preferably from about 5 to about 500 mg/kg/day when the compositions are administered as anticipated or recommended for a particular composition. Typically, fatty acid alkanolamides comprises from about 1 to about 80% of a composition, preferably from about 3 to about 50%, more preferably from about 5 to about 30%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, 40%, 45%, 50%, 55%, 60%, 70%, or 80%.

Preferably, the fatty acid alkanolamides are fatty acid ethanolamides or N-acyl fatty acid ethanolamide. Most preferably, the fatty acid alkanolamides are selected from the group consisting of N-oleoyl-ethanolamide and (Z)—(R)-9-octadecenamide, N-(2-hydroxyethyl,1-methyl).

Fatty acid alkanolamides compositions such as food, dietary, pharmaceutical, and other compositions may further comprise one or more substances such as vitamins, minerals, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat soluble vitamins as A, D, E, and K. Inulin, amino acids, enzymes, coenzymes, and the like may be useful to include in various embodiments.

In certain embodiments, fatty acid alkanolamides compositions further comprise prebiotics or probiotics. Probiotics are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Prebiotics are nondigestible food ingredients that beneficially affect host health by selectively stimulating the growth and/or activity of bacteria in the colon. Prebiotics include fructooligosaccharides (FOS), xylooligosaccharides (XOS), galactooligosaccharides (GOS), and mannooligosaccharides (typically for non-human foods such as petfoods). The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophillus, Bifidobacteria, Lactococcus, and Pediococcus. Beneficial species include Enterococcus and Saccharomyces species, e.g., Enterococcus faecium SF68. The amount of probiotics and prebiotics to be administered to the animal is determined by the skilled artisan based upon the type and nature of the prebiotic and probiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package. Probiotics and prebiotics are useful if administration of fatty acid alkanolamides upset the intestinal flora.

A skilled artisan can determine the appropriate amount of fatty acid alkanolamides, food ingredients, vitamins, minerals, probiotics, prebiotics, antioxidants, or other ingredients to be use to make a particular composition to be administered to a particular animal. Such artisan can consider the animal's species, age, size, weight, health, and the like in determining how best to formulate a particular composition comprising fatty acid alkanolamides and other ingredients. Other factors that may be considered include the type of composition (e.g., pet food composition versus dietary supplement), the desired dosage of each component, the average consumption of specific types of compositions by different animals (e.g., based on species, body weight, activity/energy demands, and the like), and the manufacturing requirements for the composition.

In a further aspect, the present invention provides kits suitable for administering fatty acid alkanolamides to an animal. The kits comprise in separate containers in a single package or in separate containers in a virtual package, as appropriate for the kit component, one or more fatty acid alkanolamides and one or more of (1) one or more ingredients suitable for consumption by an animal; (2) one or more probiotics or prebiotics; (3) one or more weight control agents; (4) instructions for how to combine fatty acid alkanolamides and other kit components to produce a composition useful for reducing lipid absorption; (5) instructions for how to use fatty acid alkanolamides and other components of the present invention such as weight control agents, particularly for the benefit of the animal; (6) a device for preparing or combining the kit components to produce a composition for administration to an animal such as a spoon or other application device; and (7) a device for administering the combined or prepared kit components to an animal such as a bowl or other container.

When the kit comprises a virtual package, the kit is limited to instructions in a virtual environment in combination with one or more physical kit components. The kit contains fatty acid alkanolamides and other components in amounts sufficient to reduce lipid absorption. Typically, fatty acid alkanolamides and the other suitable kit components are admixed just prior to consumption by an animal. The kits may contain the kit components in any of various combinations and/or mixtures. In one embodiment, the kit contains a packet containing fatty acid alkanolamides and a container of food for consumption by an animal. The kit may contain additional items such as a device for mixing fatty acid alkanolamides and other ingredients or a device for containing the admixture, e.g., a food bowl. In another embodiment, fatty acid alkanolamides are mixed with additional nutritional supplements such as vitamins and minerals that promote good health in an animal. The components are each provided in separate containers in a single package or in mixtures of various components in different packages. In preferred embodiments, the kits comprise fatty acid alkanolamides and one or more other ingredients suitable for consumption by an animal. Preferably such kits comprise instructions describing how to combine fatty acid alkanolamides with the other ingredients to form a food composition for consumption by the animal, generally by mixing fatty acid alkanolamides with the other ingredients or by applying fatty acid alkanolamides to the other ingredients, e.g. by, sprinkling fatty acid alkanolamides on a food composition.

In another aspect, the invention provides a means for communicating information about or instructions for one or more of (1) using fatty acid alkanolamides for reducing lipid absorption by an animal; (2) admixing fatty acid alkanolamides and other ingredients to produce a fatty acid alkanolamides composition such as a fatty acid alkanolamides food composition or fatty acid alkanolamides dietary supplement suitable for consumption by an animal; (3) using the kits of the present invention for the benefit of an animal, e.g., reducing body fat, reducing body weight, and reducing body weight gain in an animal; and (4) using fatty acid alkanolamides in conjunction with weight control agents for the benefit of the animal; and (5) administering fatty acid alkanolamides to an animal. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In another aspect, the invention provides methods for manufacturing a food composition comprising fatty acid alkanolamides and one or more other ingredients suitable for consumption by an animal, e.g., one or more of protein, fat, carbohydrate, fiber, vitamins, minerals, probiotics, prebiotics, weight control agents, and the like. The methods comprise admixing one or more ingredients suitable for consumption by an animal with fatty acid alkanolamides. Alternatively, the methods comprise applying fatty acid alkanolamides alone or in conjunction or combination with other ingredients onto the food composition, e.g., as a coating or topping. Fatty acid alkanolamides can be added at any time during the manufacture and/or processing of the food composition. The composition can be made according to any method suitable in the art.

In another aspect, the present invention provides a package comprising fatty acid alkanolamides and a label affixed to the package containing a word or words, picture, design, acronym, slogan, phrase, or other device, or combination thereof that indicates that the contents of the package contains an ingredient or composition suitable for reducing lipid absorption by an animal.

Typically, such device comprises the words “reduces lipid absorption”, “promotes weight loss by reducing lipid absorption”, “reduces body fat by reducing lipid absorption”, “reduces weight gain by reducing lipid absorption”, or an equivalent expression printed on the package. Any package or packaging material suitable for containing the composition is useful in the invention, e.g., a sachet, bag, box, bottle, can, pouch, and the like manufactured from paper, plastic, foil, metal, and the like. In a preferred embodiment, the package contains a food composition adapted for a particular animal such as a human, canine, or feline, as appropriate for the label, preferably a companion animal food composition for dogs or cats. In a preferred embodiment, the package is a sachet comprising fatty acid alkanolamides.

In another aspect, the invention provides for use of fatty acid alkanolamides to prepare a medicament for reducing lipid absorption by an animal. Generally, medicaments are prepared by admixing a compound or composition, i.e., fatty acid alkanolamides or fatty acid ethanolamides, with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

### EXAMPLES

The invention can be further illustrated by the following example, although it will be understood that this example is included merely for purposes of illustration and is not intended to limit the scope of the invention unless otherwise specifically indicated.

### Example 1

Adult male C57bl6j purchased at 8 week of age from Janvier Elevage (Le Genest-St-Isle, France) were individually housed and maintained ad libitum on high fat diet and water for 2 weeks. Seven mice were randomly allocated in 3 groups and fed with a high fat diet (lipids represented 50% of daily energy) during 2 weeks. For one kilogram, the composition of the high fat diet was: 235 g of casein, 201 g of saccharose, 3.5 g L-cystine, 85 g of starch, 116 g of maltodextrine, 60 g of cellulose, 12 g of vitamins mix, 51.5 g de mineral mix and 236 g of lard and 13 g of canola oil (UPAE, Jouy en Josas, France). Then, N-oleoyl-ethanolamide or (Z)—(R)-9-octadecenamide, N-(2-hydroxyethyl, 1-methyl) was added to the diet at the concentration of 100 mg/kg/bw.

The mice are kept under treatment during 4 weeks. During the nutritional intervention, daily food intake has been monitored and the mice were weighted 3 times per week. During the last week of the experiment, the mice were kept during 5 days in metabolic cages to collect urine and feces.

Lipids were extracted from feces after acidification using chloridric acid using chloroform/methanol (2:1 v/v) method. Total lipids were weighted, results were reported in mg of lipids per g of feces weight. Fat absorption was assessed using the following formula: Fat Absorption (% intake)=100*((Fat intake, g/d-fecal fat excretion, g/d)/fat intake). The results are shown in Table 1.

Referring to Table 1, compared with the control, fatty acid alkanolamides significantly reduced lipid absorption by the animals.

TABLE 1
Effect of N-oleoyl-ethanolamide (“C1”) and (Z)-(R)-
9-octadecenamide, N-(2-hydroxyethyl,1-methyl) (“C2”) Given
Orally to Mice (N = 10 per group) at 100 mg/kg of
Body Weight (mg/kg/bw) on Lipid Excretion and Absorption.

| | Lipid Excretion (mg/g of | | | Intestinal Lipid | | |
|---|---|---|---|---|---|---|
| | Feces) | | | Absorption (%) | | |
| | MV | SD | P value* | MV | SD | P value* |
| Control Group | 39.2 | 4.3 | | 97.04 | 0.30 | |
| C1 | 53.7 | 5.3 | 0.0462 | 95.61 | 0.44 | 0.0190 |
| C2 | 65.6 | 0.8 | 0.0049 | 95.05 | 0.09 | 0.0067 |

*One-way ANOVA test on the effect of N-oleoyl-ethanolamide or (Z)-(R)-9-octadecenamide, Af-(2-hydroxyethyl,1-methyl) compared to the control group. P values < 0.05 are considered to be statistically significant.

### Example 2

Example 1 was repeated except that N-oleoyl-ethanolamide as added to the diet at two different concentrations (0.1 or 1 g/kg/bw). The results are shown in Table 2.

TABLE 2
Effect of N-oleoyl-ethanolamide (0.1 g/kg/bw =
“C1” and 1 g/kg ftw = “C2”) Given
Orally to Mice (N = 10 per group) on Lipid Excretion and Absorption.

| | Lipid Excretion (mg/g of | | | Intestinal Lipid | | |
|---|---|---|---|---|---|---|
| | Feces) | | | Absorption (%) | | |
| | MV | SD | P value* | MV | SD | P value* |
| Control Group | 26.28 | 3.69 | | 98.9 | 0.17 | |
| C1 | 55.31 | 9.47 | 0.0042 | 97.4 | 0.49 | 0.0045 |
| C2 | 45.56 | 2.73 | 0.0442 | 97.76 | 0.17 | 0.024 |

*One-way ANOVA test on the effect of both concentrations of N-oleoyl-ethanolamide compared to the control group. P values < 0.05 are considered to be statistically significant.

Referring to Table 2, compared with the control, fatty acid alkanolamides significantly reduced lipid absorption by the animals.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

1. A method for reducing lipid absorption by an animal comprising administering one or more fatty acid alkanolamides to the animal in an amount effective for reducing lipid absorption by the animal.

2. The method of claim 1 wherein the fatty acid, alkanolamide is a fatty acid ethanolamide.

3. The method of claim 1 wherein the fatty acid alkanolamide is N-acyl fatty acid ethanolamide.

4. The method of claim 1 wherein the fatty acid alkanolamide is N-oleoyl-ethanolamide.

5. The method of claim 1 wherein the fatty acid alkanolamide is (Z)—(R)-9-octadecenamide, N-(2-hydroxyethyl, 1-methyl).

6. The method of claim 1 wherein the fatty acid alkanolamide is a hydrolysis-resistant fatty acid alkanolamide analog.

7. The method of claim 1 wherein fatty acid alkanolamides are administered in amounts of from about 0.1 to about 1500 mg/kg/day.

8. The method of claim 1 wherein fatty acid alkanolamides are administered to the animal in a food composition or as a dietary supplement.

9. The method of claim 8 wherein the composition is a food composition and the fatty acid alkanolamides comprise from about 1 to about 40% of the food composition.

10. The method of claim 1 further comprising administering the fatty acid alkanolamides in conjunction with one or more prebiotics or probiotics.

11. The method of claim 1 further comprising administering the fatty acid alkanolamides in conjunction with one or more weight control agents.

12. The method of claim 1 wherein the animal is a human or a companion animal.

13. The method of claim 12 wherein the companion animal is a canine or a feline.

14. A kit suitable for administering fatty acid alkanolamides to an animal comprising in separate containers in a single package or in separate containers in a virtual package, as appropriate for the kit component, one or more fatty acid alkanolamides and one or more of (1) one or more ingredients suitable for consumption by an animal; (2) one or more probiotics or prebiotics; (3) one or more weight control agents; (4) instructions for how to combine fatty acid alkanolamides and other kit components to produce a composition useful for reducing lipid absorption; (5) instructions for how to use fatty acid alkanolamides and other components of the present invention for the benefit of the animal; (6) a device for preparing or combining the kit components to produce a composition for administration to an animal; and (7) a device for administering the combined or prepared kit components to an animal.

15. The kit of claim 14 wherein the fatty acid alkanolamides are in a sachet.

16. The kit of claim 14 comprising fatty acid alkanolamides and one or more ingredients suitable for consumption by an animal.

17. The kit of claim 16 further comprising one or more weight control agents.

18. The kit of claim 14 comprising fatty acid alkanolamides and one or more weight control agents.

19. A means for communicating information about or instructions for one or more of (1) using fatty acid alkanolamides for reducing lipid absorption by an animal; (2) admixing fatty acid alkanolamides and other ingredients to produce a fatty acid alkanolamides composition; (3) using the kits of the present invention for the benefit of an animal; and (4) using fatty acid alkanolamides in conjunction with weight control agents for the benefit of the animal; and (5) administering fatty acid alkanolamides to an animal, the means comprises one or more of a physical or electronic document, digital storage media, optical storage media, audio presentation, audiovisual display, or visual display containing the information or instructions.

20. The means of claim 19 selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)
